(19) United States
(12) Patent Application Publication
(10) Pub. No.: US 2021/0158967 A1
(43) Pub. Date: May 27, 2021

(54) METHOD OF PREDICTION OF POTENTIAL HEALTH RISK

(71) Applicant: National Central University, Taoyuan City (TW)

(72) Inventors: Yi-Chiung Hsu, Taoyuan City (TW); Jia-Ching Wang, Taoyuan City (TW); Chung-Yang Sung, Taoyuan City (TW)

(73) Assignee: National Central University, Taoyuan City (TW)

(21) Appl. No.: 17/084,680

(22) Filed: Oct. 30, 2020

(30) Foreign Application Priority Data
Nov. 26, 2019 (TW) 108143024

(51) Int. Cl. G16H 50/30; G16H 50/20; G16H 50/70; G16H 50/50; G16B 20/40; G16B 25/10

(52) U.S. Cl. CPC G16H 50/30; G16H 50/20; G16B 25/10; G16H 50/50; G16B 20/40; G16H 50/70

(57) ABSTRACT

Provided herein are method of prediction of potential health risk, and particularly to a method for training artificial neural networks using biological analysis data. The method of present disclosure is characterized in the combined use of biological analysis and deep learning; in which the specific clinical data relating to the characteristic gene expression is used to train the artificial neural network to improve the accuracy of the prediction power of the artificial neural network.

### FIELD OF THE INVENTION

The present invention relates to a method of prediction of potential health risk, and particularly to a method for training artificial neural networks using biological analysis data.

### BACKGROUND OF THE INVENTION

Deep learning was the foundation of many modern AI artificial intelligence applications. Since showing breakthrough results in the field of speech recognition and image recognition, the application of deep learning in other fields has grown at an extremely fast rate. There were also considerable applications in the field of biomedicine, such as cancer detection and bioinformatics analysis.

Furthermore, with the advancement of technology and medical technology, the life span of human beings has been extended to a considerable extent, and all countries in the world have gradually become an aging population. Under this trend, the issues and challenges faced by the aging society have also received great attention. People could not be satisfied with “live to old”, but hope to “live healthy to old”. In the research of aging, there have been related researches using machine learning or deep learning to detect aging genes, but the calculation method was complicated and the accuracy was low. For the huge data that needed to be calculated, the process of screening genes was usually quite expensive, time-consuming and not efficient. In view of this, there is a great need for an improved prediction method in the technical field to improve the accuracy of prediction, reduce the time spent in a large number of biomedical testing and gene sample extraction, and be able to screen key genes more quickly and improve the deficiencies of the prior arts.

### DETAILED DESCRIPTION OF THE INVENTION

The content of the invention aims to provide a simplified summary of the disclosure so that readers have a basic understanding of the disclosure. This summary of the present invention is not a complete summary of the present disclosure, and its intention is not to point out important/key elements of the embodiments of the present invention or to define the scope of the present invention.

In order to solve the problems in the prior arts, the present invention provides a method and system for training artificial neural networks to predict whether an individual has specific gene expression characteristics.

First of all, one aspect of the present invention relates to a method of prediction of potential health risk, comprising: (1) providing a sample which comprises at least one RNA sequencing information; and (2) generating at least one physiological index and showing any deviation when compared to health people in the same chronological age group or/and model prediction; and (3) predicting the potential health risk from said physiological index or/and model prediction.

In the further embodiment, comprising: (4) tracking health conditions of source of sample.

The physiological index is BMI, blood pressure, gene expression, organ age or the combination thereof.

In the present invention, the physiological index is generated by an approach which is statistical analysis, rule-based approach, machine learning, deep learning or the combination thereof.

In an embodiment, wherein the approach is constructed, comprising: (1) providing sample which comprises RNA sequencing information; and clinical information corresponding to the RNA sequencing information; (2) using the clinical information to screen the gene expression information and analyzing the degree of variation of the plural gene expression information; (3) using statistical analysis to process the filtered gene information in the step (2) to extract at least one gene module; and (4) using at least one gene module to predict the potential health risk.

A method of constructing model for prediction of potential health risk, comprising:
(1) providing sample which comprises RNA sequencing information; and clinical information corresponding to the RNA sequencing information;
(2) using the clinical information to screen the gene expression information and analyzing the degree of variation of the plural gene expression information;
(3) using statistical analysis to process the filtered gene information in the step (2) to extract at least one gene module; and
(4) using at least one gene module to construct this type of artificial neural network for deep learning to predict the potential health risk

In a specific embodiment of the present invention, the plural pieces of gene expression information are plural pieces of FPKM (Fragments Per Kilobase of transcript per Million) information corresponding to the plural pieces of RNA sequencing information; in other words, the FPKM information are used as a feature of plural RNA sequencing information.

In an embodiment, the sample is body fluid or blood or plasma or saliva or urine.

The term “potential health risk” herein described in present invention, which means situation of individuals have and that is gene aging, medical conditions, having disease or not, the possibility of getting diseases or the combination thereof.

In the preferred embodiment, the potential health risk is gene aging.

According to an embodiment of the present invention, in the step (4), plurality of gene modules are divided into a training data set and a test data set for deep learning.

In one embodiment of the present invention, the data ratio of the training data set and the test data set is between 10:1 and 1:10. In a specific embodiment of the present invention, the data ratio of the training data set and the test data set is 4:1.

In an optional embodiment of the present invention, the clinical information is age information, gender information, disease information, symptom information, survival rate, recovery rate or the combination thereof.

The statistical analysis used in the present invention is Weighted correlation network analysis (WGCNA), Pearson product-moment correlation analysis or Spearman rank order correlation analysis. That are used to find the relationship from two factors. The factor likes gene, disease, age or others that can be compared to each other.

According to a specific embodiment of the present invention, the method is used to predict the aging gene expression characteristics of the individual, and the clinical information is age information.

In addition, in an optional manner, in step (2) of the present invention, the plural pieces of gene expression information are divided into at least five groups based on age information. In a preferred embodiment of the present invention, in step (2), the plural pieces of gene expression information are divided into at least two groups based on age information. Furthermore, the artificial neural network is classified by age information for deep learning. In addition, in the process of training artificial neural networks, the plural pieces of gene expression information are taken from non-pathological tissues and the non-pathological tissue is brain, cerebellum, lung, liver, heart or blood.

According to one embodiment of the present invention, the weighted gene co-expression analysis mainly comprises expression level cluster analysis and phenotypic correlation.

Another aspect of the present invention relates to a system used to predict whether an individual has potential health risk comprising: a computer device having a CPU processor and a memory; and an artificial neural network having an input and an output to be run on the computer device; wherein, the input can receive the data of the individual, this type of artificial neural network system can provide the output a prediction results related to the potential health risk, and this type of artificial neural network is trained by the method shown in any of the above embodiments.

After referring to the following embodiments, those with ordinary knowledge in the technical field to which the present invention belongs can easily understand the basic spirit and other purposes of the present invention, as well as the technical means and implementation aspects of the present invention.

### BRIEF DESCRIPTION OF THE DRAWINGS

In order to make the above and other objects, features, advantages and embodiments of the present invention more obvious and understandable, the description of the accompanying drawings is as follows:

FIG. 1A and FIG. 1B are flowcharts of a method for predicting aging genes according to an embodiment of the present invention;

FIG. 2 is a cluster tree formed by gene hierarchical cluster analysis of gene expression data in blood tissue of the present invention;

FIG. 3 is a diagram of the relationship between gene modules of lung tissue and age traits of the present invention;

FIG. 4 is an eigengene adjacency heatmap (eigengene adjacency heatmap) in lung tissue of the present invention;

FIG. 5 is the prediction result of the DNN training model without extracting gene modules of the present invention;

FIG. 6 is the prediction result of the DNN training model of the extracted gene module of the present invention; and

FIG. 7 is the prediction result of the DNN training model at the intersection of each tissue module and the blood tissue module of the present invention.

### EXAMPLES

In order to make the description of the present disclosure more detailed and complete, the following provides an illustrative description for the implementation aspects and specific embodiments of the present invention. This is not the only way to implement or use the specific embodiments of the present invention. The implementation manners cover the characteristics of a number of specific embodiments and the method steps and sequences used to construct and operate these specific embodiments. However, other specific embodiments can also be used to achieve the same or equal functions and sequence of steps.

Although the numerical ranges and parameters used to define the wider range of the present invention are approximate numerical values, the relevant numerical values in the specific embodiments have been presented here as accurately as possible. However, any value inherently inevitably contains the standard deviation due to individual test methods. Here, “about” usually means that the actual value is within plus or minus 10%, 5%, 1% or 0.5% of a specific value or range. Or, the term “about” means that the actual value falls within the acceptable standard error of the average value, depending on the consideration of a person with ordinary knowledge in the technical field of the present invention. Except for the experimental examples, or unless otherwise clearly stated, all ranges, quantities, values and percentages used herein (for example, to describe the amount of material, length of time, temperature, operating conditions, quantity ratio and other similar Those) have been modified by “about”. Therefore, unless otherwise stated to the contrary, the numerical parameters disclosed in this specification and the accompanying patent scope are approximate values and can be changed according to requirements. At least these numerical parameters should be understood as the indicated effective number of digits and the value obtained by applying the general carry method.

Unless the description herein, “gene expression characteristics” refers to the type of gene expression, which may be the expression level of a single gene or a characteristic formed by the expression level of multiple genes. The gene expression characteristics are related to clinical research or disease, for example, the gene expression characteristics are consistent with the aging trend, or the gene expression characteristics are consistent with the cancerization or the trend of a specific disease.

Unless the description herein, “predict” as used herein relates to statistical analysis or artificial intelligence such as machine learning or deep learning etc. They are used to predict or judge organ age difference between various organs or tissues and chronological age of sample. The results are shown as conforming and non-conforming. Non-conforming organs and tissues need to be further tracked for health conditions (health conditions).

The term “organ age” as used herein, relates to use analysis methods to determine the chronological age of each organ or tissue and sample through the genetic performance of each tissue of a healthy person. Taking Genotype-Tissue Expression (GTEx) Data set as an example but not limited to this database.

Unless otherwise defined in this specification, the scientific and technical terms used herein have the same meanings as understood and used by those with ordinary knowledge in the technical field of the present invention. In addition, without conflict with context, the singular nouns used in this specification cover the plural nouns, and the plural nouns also cover the singular nouns.

Artificial neural network is a kind of artificial intelligence that can simulate human brain activity. Generally speaking, a deep neural network comprises multiple layers of processing elements that have a weighted relationship and are related to each other to simulate the operation of brain neurons. The multilayer structure comprises an input layer, a hidden layer, and an output layer. The input of the artificial neural network is determined by the processing elements and the weight correlation between them. Therefore, a large amount of data can be used to train an artificial neural network to predict a certain gene expression characteristic of a tested individual, for example, a gene expression characteristic related to cancer or aging.

In the prior arts, machine learning or deep learning is often used to train artificial neural networks, or paired with each other to obtain better accuracy, but there are still limitations in predictive analysis.

However, the inventor of this case proposed for the first time a novel method and a system for implementing the method, combined with the method of biological analysis to train artificial neural networks for dimensionality reduction. This method can be considered in complex biological analysis experiments to consider biological characteristics and to obtain accurate analysis of experimental results.

According to an embodiment of the present invention, the system may comprise a storage device and a processor, wherein the storage device stores an artificial neural network, and when the processor loads and runs the artificial neural network, the present invention can be completed by implementing any of the methods shown in the mode. The storage device can be any type of non-volatile memory or volatile random-access memory (Random Access Memory, RAM), read-only memory (Read-Only Memory, ROM), flash memory (flash memory), hard disk (Hard Disk Drive, HDD), Solid State Drive (SSD) or similar components or a combination of the above components. Examples of the processor include, but are not limited to, a central processing unit (Central Processing Unit, CPU), or other programmable general-purpose or special-purpose microprocessor (Microprocessor), digital signal processor (Digital Signal Processor), DSP), programmable controller, Application Specific Integrated Circuit (ASIC) or other similar components or a combination of the above components.

The method suitable for the biological analysis of the present invention can use weight correlation network analysis (WGCNA) to extract gene modules related to traits or clinical characteristics, and analyze basic metabolic pathways, pathway regulation pathways or translation levels control and other biological processes, and screen out specific gene modules to achieve the effect of dimensionality reduction. In order to be able to predict individual gene expression characteristics more accurately, the method of the present invention needs to screen multiple pieces of gene expression information with specific clinical information in the process of biological analysis. In a preferred embodiment, the clinical information is a parameter related to predicting individual gene performance characteristics, including but not limited to age information, gender information, disease information, Symptom information, survival rate, or recovery rate.

In a specific embodiment, the method of the present invention can train a neural network to predict the performance characteristics of an aging gene in an individual. In this embodiment, the gene expression information is first screened by age information. Specifically, the present invention classifies gene expression data according to age, which is mainly divided into young and old age groups. Then, biological analysis methods are used to screen gene sets with relatively similar performance and the expression levels between different age groups. Gene set with significant positive and negative correlations, and then further conduct gene association network analysis and gene annotation to find the correlation between core genes and biological metabolic pathways and age, and extract the characteristic values related to age variation from them, and then do deep learning of neural network training. According to the results of this embodiment, the method of the present invention has a high accuracy rate for predicting the expression characteristics of aging genes, which means that the gene expression data extracted by the method of the present invention are highly correlated with age variation.

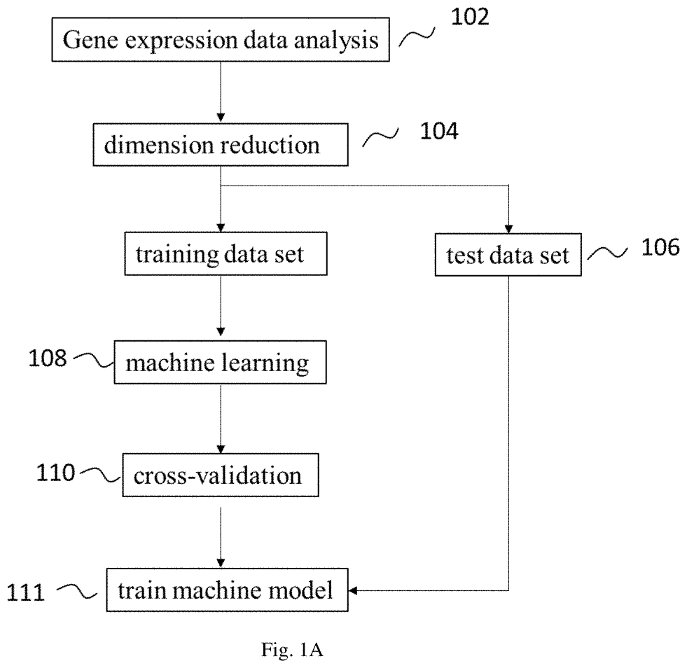

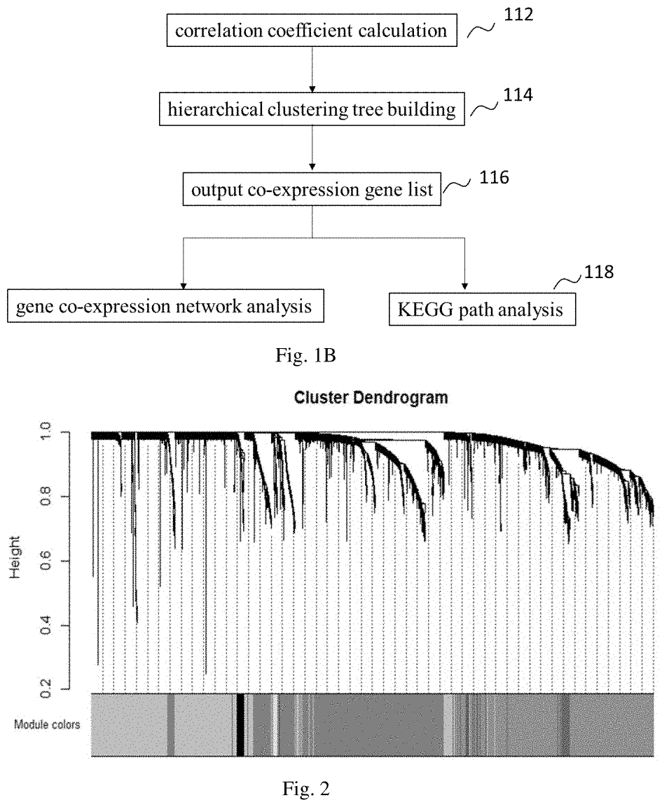

FIG. 1A and FIG. 1B are flowcharts of a method for performing machine learning to predict whether a body has aging gene expression characteristics according to a training artificial neural network shown in an embodiment of the present invention.

As shown in FIG. 1A, the present invention is different from the prior arts in that the collected gene performance data is first subjected to biological analysis (step 102). Specifically, please refer to FIG. 1B at the same time. FIG. 1B is a flowchart schematic diagram of a biological analysis process of gene expression data according to an embodiment of the present invention. In one embodiment, the gene performance data is genotype tissue performance data, which is RNA sequencing data (RNA-seq) obtained by performing next-generation sequencing on RNA. In another embodiment, the gene expression data is characterized by sequencing length, such as FPKM (Fragments Per Kilobase of transcript per Million), and classified by age information related to the gene expression. According to an optional embodiment, the gene expression data is selected from different tissues, wherein the tissues include but are not limited to brain, cerebellum, lung, liver, heart or blood. The gene performance data of each tissue is consistent with the normal distribution, and then the genes with a large degree of variation are extracted with the mean absolute error. According to an embodiment of the present invention, the number of genes with a large degree of variation may be at least 1,000, 2,000, 3,000, 4,000, or 5,000.

Next, weighted gene co-expression network analysis (WGCNA) is used to extract similar traits or clinical features between genes, and to analyze biological processes, such as basal metabolic pathways, transcriptional regulatory pathways, and translational level regulation. First, WGCNA calculates the correlation coefficient between any two genes (step 112), and can set a threshold for screening (for example, 0.9), and if it is higher than the threshold, it will be similar genes. In addition, the weighted value of the correlation coefficient is used in the analysis, and the gene correlation coefficient is taken to the power of N, so that the gene correlation in the network follows the scale-free networks.

Next, through the hierarchical clustering tree between correlation coefficients (step 114), where the clustering tree is based on the gene weighted correlation coefficients, classifies genes according to their expression mode, and classifies genes with similar mode into one module. Thousands of gene data can be divided into dozens of modules through gene expression modes (step 116), and the extracted gene modules can also be used for downstream gene co-expression network analysis or gene annotation (KEGG path analysis) (Step 118).

Please refer to FIG. 1A again, the gene modules extracted from the analysis of the gene expression data in step 102 are subjected to machine learning training, which is divided into a training data set and a test data set (step 106 and step 108), where the data ratio of the training data set to the test data set is between 10:1 and 1:10, such as 10:1, 9:1, 8:1, 7:1, 6:1, 5:1, 4:1, 3:1, 2:1, 1:1, 10:3, 5:2, 5:3, 10:7, 5:4, 10:9, 9:2, 9:4, 9:5, 3:2, 9:7, 9:8, 9:10, 8:3, 8:5, 4:3, 8:7, 8:9, 4:5, 7:10, 7:9, 7:8, 7:6, 7:5, 7:4, 7:3, 7:2, 7:1, 3:5, 2:3, 3:4, 6:7, 6:5, 6:1, 1:2 5:9, 5:8, 5:7, 5:6, 5:3, 5:2, 2:5, 4:9, 4:7, 4:5, 4:1, 3:10, 1:3, 3:8, 3:7, 1:5, 2:9, 1:4, 2:7, 2:3, 1:10, 1:9, 1:8, 1:7, 1:6, 1:5, 1:4, 1:3, or 1:2; preferably 4:1. The machine learning includes, but is not limited to, SVM, DNN, random forest, decision tree, and ridge regression. In addition, it should be noted that the present invention first adopts gene expression data analysis step 102 to perform dimensionality reduction, and can also use an autoencoder and PCA (Principal Component Analysis, PCA) to perform dimensionality reduction in combination with a conventional method (step 104).

The cross-validation method (step 110) used by the machine learning includes, but is not limited to, k-folder cross validation, kk-folder cross-validation, and least-one-out cross validation (LOOCV), 10-fold cross validation. In one embodiment, the cross-validation is 10-fold cross-validation. Finally, training the machine model 111 uses to predict the expression characteristics of aging genes. According to other embodiments of the present invention, the independent data verification, loss function and activation function comparison of the machine model training can be selected based on the general experience and actual use requirements of persons with general knowledge in the relevant technical field.

The cross-validation method (step 110) used by the machine learning includes, but is not limited to, k-folder cross validation, kk-folder cross-validation, and least-one-out cross validation (LOOCV), 10-fold cross validation. In one embodiment, the cross-validation is 10-fold cross-validation. Finally, the machine model 111 is trained to predict the expression characteristics of aging genes. According to other embodiments of the present invention, the independent data verification, loss function and activation function comparison of the machine model training can be selected based on the general experience and actual use requirements of persons with general knowledge in the relevant technical field.

In addition, the software suitable for the machine learning of the present invention can be deep learning software Anaconda, Spyder, WEKA. In addition, the biometric analysis software suitable for use in the present invention can be Cytoscape or R-studio.

A number of experimental examples are presented below to illustrate certain aspects of the present invention, in order to facilitate those skilled in the art to which the present invention pertains to practice the present invention, and these experimental examples should not be regarded as limiting the scope of the present invention. It is believed that those skilled in the art can fully utilize and practice the present invention without excessive interpretation after reading the description presented here. The full text of all published documents cited here are regarded as part of this specification.

### Experimental Example 1

Gene Expression Data

The gene expression data used in this experimental example was from the database dbGAP accession phs000424.v7.p2 in GTEx Portal (Genotype-Tissue Expression). In this example, the genetic data came from 714 donors. LDACC (Rhe Laboratory, Data Analysis and Coordinating Center) performs nucleic acid extraction and quality evaluation on RNA-seq samples. To measure gene expression, LDACC used microarrays and RNA next-generation sequencing for analysis. In this experimental example, brain, lung, heart, liver, and blood tissues were used as the analysis targets. The number of samples for each tissue was 173, 427, 303, 175, and 407, respectively. The RNA-seq expression of these tissues was characterized by FPKM value and classified by age information. Please refer to Table 1 for the distribution of the five tissues and their corresponding age data.

TABLE 1

| | Cerebellum | Lung | Heart | Liver | Blood |
|---|---|---|---|---|---|
| 20-29 | 7 | 27 | 21 | 7 | 34 |
| years old | | | | | |
| 30-39 | 4 | 30 | 18 | 10 | 34 |
| years old | | | | | |
| 40-49 | 17 | 76 | 50 | 28 | 72 |
| years old | | | | | |
| 50-59 | 58 | 145 | 111 | 65 | 130 |
| years old | | | | | |
| 60-69 | 82 | 139 | 96 | 62 | 132 |
| years old | | | | | |
| 70-79 | 5 | 10 | 7 | 3 | 5 |
| years old | | | | | |
| Total | 173 | 427 | 175 | 303 | 407 |

After the gene expression data of the present invention was processed, it was divided into a training data set and a test data set (data ratio: 8:2) for prediction. Please refer to Table 2 for the neural network parameters used in the present invention.

TABLE 2

| DNN | |
|---|---|
| Input layer | 15714 |
| Input dim | 506 |
| Hidden layer | 10000, 1000, 100 |
| Output layer | 2 |
| Learning rate | 0.001 |
| Autoencoder | |
| Input layer | 15714 |
| Bottleneck layer | 300 |
| Learning rate | 0.001 |

Data Preprocessing

After making the gene expression data of each tissue consistent with the normal distribution, the first 5000 genes with large degree of variation were extracted with the mean absolute error.

Gene Hierarchical Cluster Analysis

Here, using WGCNA calculations and determining the relevant values through soft-thresholding, the best parameter beta value was 7, the codes are shown in Table 3, and are classified by gene phenotype. Then clustering is based on gene phenotype and similarity, and closely related genes were clustered into one module. Therefore, 5000 genes were classified into several modules.

TABLE 3
WGCNA

powers = c(c(1:10), seq(from = 12, to=30, by=2)
sft = pickSoftThreshold(datExpr, powerVector = powers, verbose = 5)
soft-thresholding powers(best_beta) = 7

The classified plural modules were basically similar in function to each module, and therefore, genes within the same module could be regarded as similar or related. FIG. 2 is a cluster tree formed by gene hierarchical cluster analysis of gene expression data in blood tissues. The data distribution of gene hierarchical clustering in each color block was shown in Table 4 below.

TABLE 4

| | | | | | | | Blue | | |
|---|---|---|---|---|---|---|---|---|---|
| Black | Blue | Brown | Green | Gray | Pink | Red | Green | Yellow | Total |
| 62 | 1283 | 190 | 155 | 1493 | 38 | 71 | 1546 | 162 | 5000 |

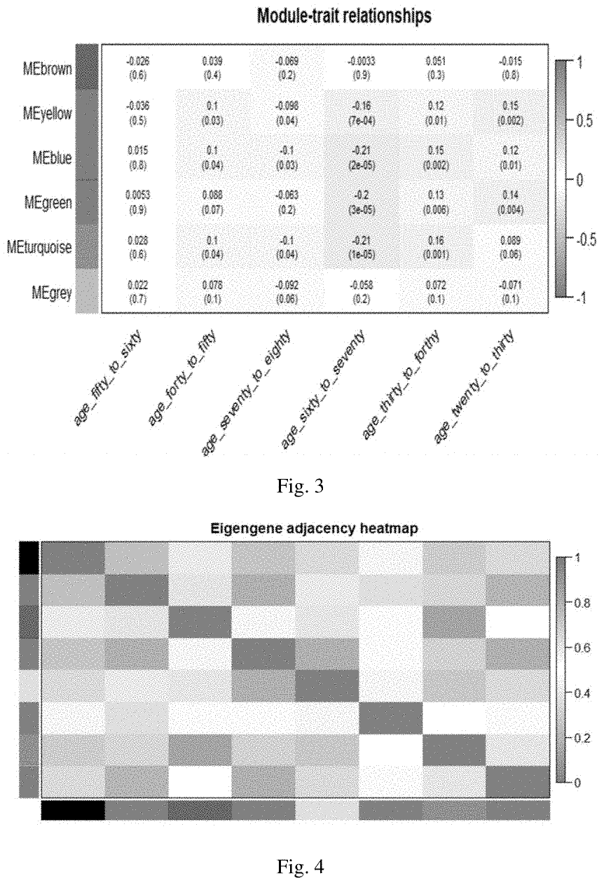

The gene module trait analysis was then used to screen out genes with large degree of variation between age groups. In principle, the difference between positive and negative correlations was greater than 0.2 as a benchmark, please refer to FIG. 3. Taking lung tissue as an example, FIG. 3 showed the relationship between gene modules of lung tissue and age traits. As shown in the results, the green (MEgreen) trait in the figure was a gene module related to lung tissue. In terms of the distribution of age groups, the lower age group was positively correlated (red), and the high age group is negatively correlated (green). Therefore, the green color was extracted. There were 114 gene samples in the green module (MEgreen).

In addition, the association in the gene modules is analyzed. In the analysis of related gene modules, it could compare the correlation between any two modules in the same tissue to explore the interaction between different modules. Also take the lung tissue as an example, where the characteristic genes in the lung tissue were adjacent to the heat map as shown in FIG. 4.

Deep Learning

The present invention uses Multi-Layer Perception (MLP) as a deep neuron network. The operating principle of MLP is that given labeled training data set x={x₁, x₂, . . . , xₙ} and combined with labeled target data by supervised learning method to train perceptron. In the training process, it often applies Back-propagation to minimize the training error and make input value x approach target value d, the function recited as follows:

y=f(W⁽²⁾(f(W⁽¹⁾x+b⁽¹⁾))+b⁽²⁾) (1)

where W⁽¹⁾ is weight matrix, b⁽¹⁾ is offset, f is activation function. The parameter from input layer to hidden layer is (W⁽¹⁾,b⁽¹⁾), and the part from hidden layer to output layer. This stage will make hidden layer x⁽¹⁾ map to output layer y=[y₁, y₂, . . . , yₖ]^{T}. W⁽²⁾ is a k×m weight matrix, b (2) is decoding offset and f is as just mentioned. the parameter from hidden layer to output layer is (W⁽²⁾,b⁽²⁾).

Data Normalization

The way of data normalization is Group normalization in the instant invention which replaces Batch normalization that often be used in neuron network. In order to maintain good results in smaller batch size. Therefore, the purpose is transform and reconstruct the data and introduce two learnable parameter γ and β, the function is

[symbol]

= [symbol] +β (2)

Where [symbol] is

$${\hat{𝓍}}_{𝒾} = \frac{1}{{σ}_{𝒾}} ( {𝓍}_{𝒾} - {μ}_{𝒾} ) \qquad ( 3 )$$

where x is characters of the data and then divide [symbol] into three vector are respectively N, C and F. Where N is the batch axis, C is the channel axis, and F is feature axis. If [symbol] =( [symbol] ), The calculation formula for the value μ and the variance σ is as follows:

$$\begin{matrix}𝓀 = \frac{{i}_{C}}{C / G} & ( 4 ) \\ {μ}_{i} = \frac{1}{𝓂} {Σ}_{i = 1}^{𝓀} {𝓍}_{i} & ( 5 ) \\ { }^{{{σ}_{i}}^{2} = \frac{1}{𝓂} {{Σ}_{i = 1}^{𝓀} ( {𝓍}_{i} - {μ}_{i} )}^{2} + ϵ} & ( 6 )\end{matrix}$$

where ϵ is a small constant, m is the size of the set, G is the number of groups, and the group is a hyper parameter that is self-defined. C/G is the channel of each group. Therefore, the method of normalization is originally normalized in each batch and changed to normalization across channels, which allows training with smaller batch size and achieves the expected effect for normalization.

Activation Function

Traditionally, ReLU (Rectified Linear Unit) is commonly used activation function in deep learning models. In the present invention, the activation function which is different from other known machine learning model is apply SeLu (Scaled exponential linear unit). The function is

$$f ( 𝓍 ) = \{ \begin{matrix}0 , & for 𝓍 < 0 \\ 𝓍 , & for 𝓍 \geq 0\end{matrix} \} \qquad ( 7 )$$

The advance of SeLu is having the faster calculating speed and conducing to back propagate. But in the negative part, there may be neurons cannot be update forever (the negative part, the gradient is 0), and another side that is greater than zero, the data will not be amplitude compressed so that the gradient cannot expand continuously.

In the preferred embodiment, the parameter λ is 1.050700987355480493419, α is 1.673263242354377284817. When λ is positive number greater than 1, it can reduce and prevent the gradient rising endlessly. On the other hand, λ is too small that will increase the gradient and prevent from disappearing. In this way ensuring the performing of normalization of each layer in deep neuron network.

Generally speaking, in the process of traditional machine learning, over fitting is prone to occur. There are three aspects to this situation: applying a too complex model, too much data noise and insufficient training data, so that the output can be applied to the training data set with complexity the model, but it is not suitable for the test data set. Therefore, in the present invention, the auto encoder is used to reduce the data dimension to avoid the occurrence of overfitting. Of course, if applying with support vector machine model (SVM), it also avoids the occurrence of overfitting.

Auto-Encoder

The architecture of auto-encoder is extended from perceptron which is a non-supervised learning method. The most difference between auto-encoder and perceptron is back-propagation. Auto-encoder hope to output y value can close to input x value, so it does not need target value d.

The architecture of auto-encoder has an input layer (dimension is n), a hidden layer (dimension is m) and an output layer. The training part is divided into two parts: encoding (input layer to hidden layer) and decoding (hidden layer to output layer). Where the encoding part maps the input layer data to the hidden layer and the decoding part must be restored to the original signal. Therefore, the weight of the decoding part is directly the transposition of the encoding part.

In order to achieve effective training, sparse auto encoder (SAE) is particularly used in the implementation of the present invention. If the output value of a neuron is close to 1, it is considered that the neuron is activated; and if the output value is close to 0, the neuron is considered to be inhibited. Therefore, the limitation of sparsity is like that most of the time the neuron is inhibited. The relevant formula is as follows:

$$E = \frac{1}{𝓃} D ( x , \hat{x} ) + \frac{λ}{2} {\sum }_{l} {\sum }_{i} {\sum }_{j} {( {W}_{j i}^{( l )} )}^{2} + {\sum }_{j} K L ( ρ ‖ {\hat{ρ}}_{j} ) \qquad ( 8 )$$

Where D(. , .) is the measure of the difference between the two vectors, l is the index value of the number of layers, i and j are the index value of the neuron number of the layers before and after the weight matrix connection respectively. λ is the coefficient of the normalization.

In addition,

$${\hat{ρ}}_{j} = \frac{1}{𝓂} {{Σ}^{𝓂}}_{i = 1} [ {𝒽}_{j} ( {𝓍}^{( i )} ) ] ,$$

i is the index value of the number of records in the data (total number of records is m), j is the index value of the number of neurons in the hidden layer, hⱼ(x⁽ⁱ⁾) and is the excitation value of the i neuron in the hidden layer under the j data.

Support Vector Machine (SVM)

Support vector machine is a supervised learning method in machine learning. It is a simple two-class classifier that can be applied to regression analysis and statistical classification. The best advantage is that it still has lower error after the verifying test samples by decision rule which comes from limited and small training samples, so this method can play its strengths in solving a small number of samples, nonlinear and high-dimensional pattern recognition problems.

Assuming that the training data set contains N pieces of data x₁, x₂, . . . xₙ, each observation xₙ, nϵ{1, . . . , N} has a corresponding tₙϵ{−1,1} representing its category, because I only want Obtain the hyperplane when the classification is correct, so tₙy(xₙ)>0, then the distance from xₙ to the hyperplane is as follows:

$$\frac{{t}_{n} y ( {x}_{n} )}{‖ w ‖} = \frac{{t}_{n} ( {w}^{T} φ ( {x}_{n} ) + b )}{‖ w ‖} \qquad ( 9 )$$

Where ϕ(x) is the conversion of projection-observing x to a fixed feature space, b is a constant, used to represent the deviation (Bias).

Furthermore, the formula that used to calculate maximizes from xₙ to the hyperplane,

$${arg max}_{w , b} \{ \frac{1}{‖ w ‖} {min}_{n} [ {t}_{n} ( {w}^{T} φ ( {x}_{n} ) + b ) ] \} \qquad ( 10 )$$

Because formula (10) is complexity and it is not easy to get answer, so simplifying into following formula:

$${arg min}_{w} \frac{1}{2} {‖ w ‖}^{2} \\ subject to {t}_{n} ( {w}^{T} φ ( {x}_{n} ) + b ) \geq 1 , \begin{matrix}n = 1 , ... , N & \end{matrix} \qquad ( 11 )$$

Using the Lagrangian Multiplier Method to formula (11), and get two conditions as follows:

$$\begin{matrix}w = {\sum }_{n = 1}^{N} {a}_{n} {t}_{n} φ ( {x}_{n} ) & ( 12 ) \\ 0 = {\sum }_{n = 1}^{N} {a}_{n} {t}_{n} & ( 13 )\end{matrix}$$

When aₙ is Lagrange Multipliersm, the predicted value of a new test data x from the following formula:

$$y ( x ) = {\sum }_{n = 1}^{N} {a}_{n} {t}_{n} k ( x , {x}_{n} ) + b \qquad ( 14 )$$

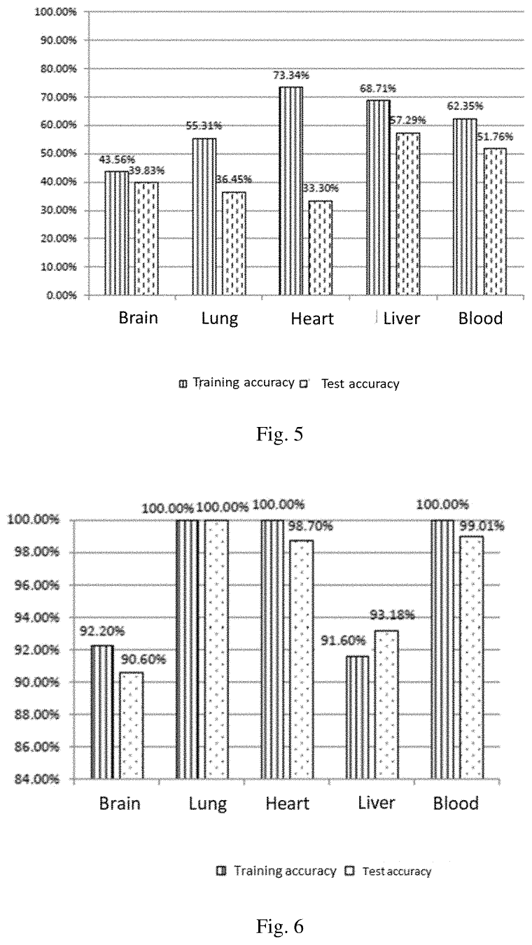

From the results of the above analysis using WGCNA, five potential tissue trait gene modules were selected, and six age categories were used for deep learning training. According to the gene set, when the number of tissue samples was fixed, the number of genes in the training data set decreased. This experimental example achieved the effect of dimensionality reduction with target selection and age-related modules. Therefore, when the DNN prediction was done by dividing into six groups of ages, the accuracy was higher than that of the gene expression information without WGCNA experiment. For the results, please refer to Table 5 and Table 6, and FIGS. 5 and 6.

Table 5 is the GTEx gene expression data set that had not been analyzed and processed by WGCNA. Please refer to FIG. 6 for the prediction results of this method.

TABLE 5

| Tissue | sample | gene number | gene expression data set |
|---|---|---|---|
| Brain | 173 | 16248 | 2810904 |
| Lung | 427 | 15714 | 6709878 |
| Heart | 303 | 16223 | 4915569 |
| Liver | 175 | 16223 | 2839025 |
| Blood | 407 | 16575 | 6746025 |

Table 6 shows the expression data of the extracted five tissue gene modules.

| Tissue | sample | gene number | gene expression data set |
|---|---|---|---|
| Brain | 173 | 134 | 23182 |
| Lung | 427 | 117 | 49959 |
| Heart | 303 | 506 | 153318 |
| Liver | 175 | 83 | 14525 |
| Blood | 407 | 1545 | 628815 |

As shown in FIG. 6, the method of the present invention was used to perform biological analysis first, and the extracted gene expression data was classified by age to perform deep learning for these six categories (one category for every 10 years). The results showed that the prediction accuracy obtained from five tissues is higher than 90%.

In order to further limit the scope of gene expression data, based on the correlation between the blood tissue gene module and other tissue modules, the gene module and the blood tissue module were intersected to obtain the following data:

TABLE 7

| | | gene | gene expression |
|---|---|---|---|
| Tissue | sample | number | data set |
| Cerebellum | 173 | 5 | 865 |
| Lung | 427 | 4 | 1708 |
| Heart | 303 | 15 | 4545 |
| Liver | 175 | 4 | 700 |

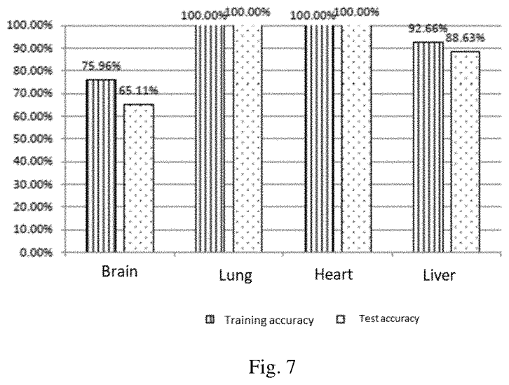

DNN training was performed on the gene expression data in six age categories, and the results were shown in FIG. 7. The results showed that, except for the slightly lower accuracy of the cerebellum, the accuracy of the lung, heart, and liver tissues are all higher than 90%, which represents the correlation between the gene expression data set and age, and the variation is related.

In order to present the advantages of the present invention, Table 8 shows the average accuracy and recall rate of DNN training for six ages in the above three tests.

TABLE 8

| | | | gene module and |
|---|---|---|---|
| | | extraction gene module | blood gene module |
| | DNN | (WGCNA) + DNN | intersection + DNN |
| Precision | 0.5306 | 0.8836 | 0.8544 |
| Recall | 0.4719 | 0.9206 | 0.8361 |
| F-Score | 0.5174 | 0.9467 | 0.8732 |

According to Table 8, the method of the present invention used WGCNA to perform biological analysis and extracted gene modules, and then used six age groups to perform DNN prediction. The results were better in accuracy, recall and F-score. It can be seen that the method proposed by the present invention can improve the accuracy of machine learning prediction. In addition, it should be noted that the present invention maintains a high accuracy rate in the complex gene expression data and the prediction model training divided into six age groups and multiple categories.

### Experimental Example 2

The model was constructed to predict potential health risk while comparing to health people in the same chronological age group. For example, the number of samples were divided into six age ranges. The training data of the normal organ age target category was the samples in the first range of age, and the training data of the abnormal organ age target category was the samples in the second, third, fourth, fifth, and sixth age ranges. The model was trained by process as described earlier. When the model used for subjects in the first range of age, it was used to determine whether their organ age is normal or abnormal.

As mentioned above, the training data of the normal organ age target category was the samples in the second range of age, and the training data of the abnormal organ age target category was the samples in the first, third, fourth, fifth, and sixth age ranges. The model was trained through above process for subjects in the second range of age, it was used to determine whether their organ age is normal or abnormal.

If the result from the model prediction was abnormal, it would be further identified which organ has abnormal gene expression while comparing to health people in the same chronological age group. The purpose of finding which gene expression of organ is abnormal was done by experimental example 1. It is helpful and precise for related personnel to tracking health condition.

Although the specific embodiments of the present invention are disclosed in the above embodiments, they are not intended to limit the present invention. Those with ordinary knowledge in the technical field to which the present invention belongs will not depart from the principles and spirit of the present invention. Below, various changes and modifications can be made to it, so the protection scope of the present invention should be defined by the accompanying patent application.

What is claimed is:

1. A method of prediction of potential health risk, comprising:
(1) providing a sample which comprises at least one RNA sequencing information; and
(2) generating at least one physiological index and showing any deviation when compared to health people in the same chronological age group or/and model prediction; and
(3) predicting the potential health risk from said physiological index or/and model prediction.

2. The method of claim 1, further comprising: (4) tracking health conditions of source of sample.

3. The method of claim 1, wherein the sample is cell, body fluid, blood, plasma, saliva, urine, tissue, pieces of organ or the combination thereof.

4. The method of claim 1, wherein the potential health risk is gene aging, medical conditions, having disease or not, the possibility of getting diseases or the combination thereof.

5. The method of claim 1, wherein the physiological index is organ age.

6. The method of claim 1, wherein the physiological index is generated by an approach which is statistical analysis, rule-based approach, machine learning, deep learning or the combination thereof.

7. The method of claim 1, wherein at least one RNA sequencing information is taken from non-pathological tissue and the non-pathological tissue is brain, cerebellum, lung, liver, heart or blood.

8. The method of claim 6, wherein the approach is constructed, comprising:
(1) providing sample which comprises RNA sequencing information; and clinical information corresponding to the RNA sequencing information;
(2) using the clinical information to screen the gene expression information and analyzing the degree of variation of the plural gene expression information;
(3) using statistical analysis to process the filtered gene information in the step (2) to extract at least one gene module; and
(4) using at least one gene module to predict the potential health risk.

9. A method of constructing model for prediction of potential health risk, comprising:
(1) providing sample which comprises RNA sequencing information;
and clinical information corresponding to the RNA sequencing information;
(2) using the clinical information to screen the gene expression information and analyzing the degree of variation of the plural gene expression information;
(3) using statistical analysis to process the filtered gene information in the step (2) to extract at least one gene module; and
(4) using at least one gene module to construct this type of artificial neural network for deep learning to predict the potential health risk.

10. The method of claim 9, wherein at least one gene expression information is at least one of FPKM (Fragments Per Kilobase of transcript per Million) information corresponding to at least one RNA sequencing information.

11. The method of claim 9, wherein the clinical information is age information, gender information, disease information, symptom information, survival rate, recovery rate or the combination thereof.

12. The method of claim 11, wherein the clinical information is age information, and the gene expression characteristic is an aging gene expression characteristic.

13. The method of claim 9, wherein in the step (2), the gene expression information is divided into at least two groups based on the age information.

14. The method of claim 9, wherein in the step (3), the statistical analysis is weighted correlation network analysis, Pearson product-moment correlation analysis or Spearman rank order correlation analysis.

15. The method of claim 14, wherein the statistical analysis is weighted correlation network analysis which comprises expression cluster analysis and phenotypic association.

16. The method of claim 9, wherein in the step (4), at least one gene module is divided into a training data set and a test data set for deep learning.
